(19) United States
(12) Patent Application Publication
(10) Pub. No.: US 2018/0228193 A1
(43) Pub. Date: Aug. 16, 2018

(54) METHOD FOR THE PRODUCTION OF A MICROALGAL BIOMASS OF OPTIMISED SENSORY QUALITY

(71) Applicant: Corbion Biotech, Inc., South San Francisco, CA (US)

(72) Inventors: Amandine Druon, Lille (FR); Samuel Patinier, Quesnoy-Sur-Deule (FR)

(21) Appl. No.: 15/945,651

(22) Filed: Apr. 4, 2018

Related U.S. Application Data
(63) Continuation of application No. 14/900,681, filed on Dec. 22, 2015, now abandoned.

(30) Foreign Application Priority Data
Jun. 26, 2013 (FR) 1356110

(51) Int. Cl. A23L 29/00; C12N 1/12; A61K 36/05; A23K 10/16; A23L 17/60

(52) U.S. Cl. CPC A23L 29/065; C12N 1/12; A23V 2002/00; A23K 10/16; A23L 17/60; A61K 36/05

(57) ABSTRACT

The methods disclosed herein relate to methods for conditioning a biomass of microalgae of the genus Chlorella, produced by fermentation under heterotrophic conditions and in the absence of light for the preparation of a flour having an optimized sensory profile.

The present invention relates to a novel method for producing a biomass of microalgae of the Chlorella genus which allows the preparation of a flour having an optimized sensory profile. The present invention therefore permits the incorporation of this microalgal flour into food formulations without generating undesirable flavors.

### PRESENTATION OF THE PRIOR ART

Historically requiring “only water and sunlight” to grow, algae have for a long time been considered to be a source of food.

There are several species of algae that can be used in food, most being “macroalgae” such as kelp, sea lettuce (Ulva lactuca) and red algae of the type Porphyra (cultured in Japan) or “dulse” (Palmaria palmata).

However, in addition to these macroalgae, there are also other algal sources represented by the “microalgae”, i.e. photosynthetic or non-photosynthetic single-cell microscopic algae, of marine or non-marine origin, cultured for their applications in biofuels or food.

For example, spirulina (Arthrospira platensis) is cultured in open lagoons (under phototrophic conditions) for use as a food supplement or incorporated in small amounts into confectionery products or drinks (generally less than 0.5% weight/weight).

Other lipid-rich microalgae, including certain species of Chlorella, are also very popular in Asian countries as food supplements (mention is made of the omega-3-producing microalgae of the Crypthecodinium or Schizochytrium genus).

The production and the use of the flour of microalgae of Chlorella type are, for example, described in documents WO 2010/120923 and WO 2010/045368.

The oil fraction of the microalgal flour, which may be composed essentially of monounsaturated oils, may provide nutritional and health advantages compared with the saturated, hydrogenated and polyunsaturated oils often found in conventional food products.

When it is desired to industrially produce microalgal flour powders from the biomass of said microalgae, considerable difficulties remain, not only from the technological point of view, but also from the point of view of the sensory profile of the flours produced.

Indeed, while algal powders for example produced with algae photosynthetically cultured in exterior ponds or by photobioreactors are commercially available, they have a dark green color (associated with chlorophyll) and a strong, unpleasant taste.

Even formulated in food products or as nutritional supplements, these algal powders always give this visually unattractive green color to the food product or to the nutritional supplement and have an unpleasant fishy taste or the savor of marine algae.

Moreover, it is known that certain species of blue algae naturally produce odorous chemical molecules such as geosmin (trans-1,10-dimethyl-trans-9-decalol) or MIB (2-methylisobomeol), generating earthy or musty odors.

As for chlorellae, the descriptor commonly accepted in this field is the taste of “green tea”, slightly similar to other green vegetable powders such as powdered green barley or powdered green wheat, the taste being attributed to its high chlorophyll content.

Their savor is usually masked only when they are mixed with vegetables with a strong savor or citrus fruit juices.

There is therefore still an unsatisfied need to have a method for preparing biomass of microalgae of the Chlorella genus of suitable organoleptic quality allowing the use of the flour prepared from said microalgae in more numerous and diversified food products. Moreover, still in the spirit of industrial optimization, a method which reproducibly provides microalgae of the Chlorella genus of reproducible organoleptic quality would be very advantageous.

### SUBJECT OF THE INVENTION

In order to devise the method of the invention, the applicant company first chose to form a sensory panel in order to evaluate the sensory properties of various batches of Chlorella protothecoides biomass flour. The sensory description of production batches then allows the identification of the key steps of the method which will allow the production of microalgal biomass flour of organoleptic quality in accordance with expectations, and reproducibly.

In carrying out its production of the microalgal biomass by fermentation under heterotrophic conditions and in the absence of light, as will be exemplified hereinafter, the applicant company therefore varied the various biomass fermentation and treatment parameters in order to generate these various batches. The applicant company finally succeeded in demonstrating a correlation between the sensory note given by the sensory panel to each batch produced and some of the conditions for carrying out the method for producing said batches.

This correlation then enabled the applicant company to select the parameters for carrying out the biomass fermentation and treatment which, taken alone or in combination, guarantee the production of Chlorella biomass having an optimized sensory profile.

The applicant company then proposed a production method for conditioning a biomass of microalgae of the Chlorella genus, preferably Chlorella protothecoides, for the preparation of a flour having an optimized sensory profile.

The present invention therefore relates to a method for conditioning a biomass of microalgae of the Chlorella genus, preferably Chlorella protothecoides, produced under heterotrophic conditions and in the absence of light for the preparation of a flour having an optimized sensory profile, the conditioning method being characterized in that it comprises the steps of:
collection of the biomass directly at the end of fermentation, and storage for less than 8 hours before conditioning thereof,
high temperature/short time (HTST) heat treatment of the thus recovered and stored biomass, for 30 seconds to 1 min 30, at a temperature below 70° C., and
washing of the biomass with at most 3 volumes of water per volume of biomass.

Preferably, the storage time for the biomass before it is conditioned and milled is less than 3 hours, preferably less than 1 hour.

Preferably, the HTST heat treatment is carried out for 1 minute at a temperature of between 60 and 68° C., preferably 65° C.±2° C., in particular 65° C.

Preferably, the biomass is washed with one volume of water per volume of biomass.

In one preferred embodiment, the HTST treatment is carried out before the step of washing the biomass.

In one preferred embodiment, the conditioned biomass was obtained by fermentation of the microalga of the Chlorella genus, preferably Chlorella protothecoides, at an initial pH between 6.5 and 7, preferably 6.8, and with regulation of the fermentation pH at a value of between 6.5 and 7, preferably at a value of 6.8.

The present invention also relates to a method for producing a biomass of microalgae of the Chlorella genus, preferably Chlorella protothecoides, for the preparation of a flour having an optimized sensory profile, comprising:
the production of a biomass by fermentation of microalgae of the Chlorella genus, preferably Chlorella protothecoides, under heterotrophic conditions and in the absence of light, the initial pH of the fermentation and the regulation of the pH during fermentation being fixed at a value of between 6.5 and 7, preferably at a value of 6.8; and
the conditioning of the biomass by means of a method as described in the present document.

Finally, the present invention also relates to a method for preparing a flour of microalgae of the Chlorella genus, preferably Chlorella protothecoides, having an optimized sensory profile, comprising:
the conditioning of the biomass by means of a method as described in the present document;
the milling of the conditioned biomass; and
the drying of the milled biomass.

Optionally, the method also comprises, prior to the conditioning, the production of a biomass by fermentation of microalgae of the Chlorella genus, preferably Chlorella protothecoides, under heterotrophic conditions and in the absence of light, the initial pH of the fermentation and the regulation of the pH during fermentation being fixed at a value of between 6.5 and 7, preferably at a value of 6.8.

### DETAILED DESCRIPTION OF THE INVENTION

For the purposes of the invention, a microalgal flour has an “optimized sensory profile” when its evaluation by a sensory panel in a food formulation or tasting formulation (for example, ice cream or tasting recipe as described herein) concludes that there is an absence of off-notes which impair the organoleptic quality of said food formulations containing this microalgal flour.

These off-notes can be associated with the presence of undesirable specific odorous and/or aromatic molecules which are characterized by a perception threshold corresponding to the minimum value of the sensory stimulus required to arouse a sensation.

The “optimized sensory profile” is then reflected by a sensory panel by obtaining the best scores on a scale evaluation of the 4 sensory criteria (appearance, texture, savors and flavors).

For the purposes of the present invention, the term “microalgal flour” should be understood in its broadest interpretation and as denoting, for example, a composition comprising a plurality of particles of microalgal biomass. The microalgal biomass is derived from microalgal cells, which may be whole or lyzed, or a mixture of whole and lyzed cells.

A certain number of prior art documents, such as international patent application WO 2010/120923, describe methods for the production and use in food of Chlorella microalgal biomass.

The microalgae of which it is a question in the present invention are microalgae of the Chlorella genus, more particularly Chlorella protothecoides, even more particularly Chlorella deprived of chlorophyll pigments, by any method known per se to those skilled in the art (either because the culture is carried out in the dark under certain operating conditions well known to those skilled in the art, or because the strain has been mutated so as to no longer produce these pigments).

The fermentative method described in this patent application WO 2010/120923 thus allows the production of a certain number of microalgal flours of variable sensory quality, if the conditions for fermentation and treatment of the biomass produced are varied.

The applicant company thus chose to vary and analyze the impact of the following parameters:
initial pH of the fermentation medium,
pH during fermentation,
acidification during harvesting of the fermentation must,
heat treatment of the biomass (treatment referred to as HTST),
washing of the biomass,
milling of the biomass,
adjustment of the pH,
pasteurization,
drying, and
storage of the flour thus obtained.

As has been exemplified hereinafter, the key steps of the method for conditioning the biomass so as to optimize the sensory profile of microalgal flours are the following:
collection of the biomass directly at the end of fermentation, and storage for less than 8 hours before conditioning thereof preceding milling thereof,
HTST treatment of the thus recovered and stored biomass, carried out for between 30 seconds and 1 min 30, at a temperature below 70° C.,
washing of the HTST-treated biomass with at most 3 volumes of water per volume of biomass.

Thus, the biomass is collected as rapidly as possible so as to undergo the subsequent heat treatment and/or washing steps. It was determined that the storage must be as short as possible. Preferably, the storage lasts less than 8, 7, 6, 5, 4, 3, 2 or 1 hour(s). Preferably, the storage time for the biomass before it is conditioned and milled is less than 3 hours, preferably less than 1 hour. Ideally, the storage step is absent and the biomass collected is directly subjected to the subsequent heat treatment and/or washing steps.

It was also determined that the HTST heat treatment also had an impact on the sensory profile and therefore the organoleptic quality of the microalgal flour. Thus, the temperature is preferably below or equal to 70° C. and above 50° C. It can be between 55 and 70° C., preferably between 60 and 68° C., preferably 65° C. The treatment time is preferably 1 minute.

It was also shown that the washing could be optimized while at the same time improving the sensory profile and therefore the organoleptic quality of the microalgal flour. Thus, preferably, the biomass is washed with 3, 2.5, 2, 1.5 or 1 volume(s) of water for one volume of biomass. In one embodiment, one volume of water will be used for one volume of biomass.

Finally, it was shown that the order of the conditioning steps had an impact on the sensory profile and therefore the organoleptic quality of the microalgal flour. In particular, it is preferable to carry out the HTST heat treatment before the step of washing the biomass.

The conditioned microalgal biomass is a biomass prepared by fermentation, under heterotrophic conditions and in the absence of light, of a microalga of the Chlorella genus, preferably Chlorella protothecoides.

Optionally, the microalgae used can be chosen, non-exhaustively, from Chlorella protothecoides, Chlorella kessleri, Chlorella minutissima, Chlorella sp., Chlorella sorokiniama, Chlorella luteoviridis, Chlorella vulgaris, Chlorella reisiglii, Chlorella ellipsoidea, Chlorella saccarophila, Parachlorella kessleri, Parachlorella beijerinkii, Prototheca stagnora and Prototheca moriformis.

Preferably, the microalgae used according to the invention belong to the Chlorella protothecoides species. According to this preferred mode, the algae intended for the production of the microalgal flour have the GRAS status. The GRAS (Generally Recognized As Safe) concept, created in 1958 by the Food and Drug Administration (FDA), allows the regulation of substances or extracts added to foods and which are considered to be harmless by a panel of experts.

The fermentation conditions are well known to those skilled in the art. The appropriate culture conditions to be used are in particular described in the article by Ikuro Shihira-Ishikawa and Eiji Hase, “Nutritional Control of Cell Pigmentation in Chlorella protothecoides with special reference to the degeneration of chloroplast induced by glucose”, Plant and Cell Physiology, 5, 1964.

This article describes in particular that all the color grades can be produced by Chlorella protothecoides (colorless, yellow, yellowish green, and green) by varying the nitrogen and carbon sources and ratios. In particular, “washed-out” and “colorless” cells are obtained using culture media which are glucose-rich and nitrogen-poor.

The distinction between colorless cells and yellow cells is made in this article. Furthermore, the washed-out cells cultured in excess glucose and limited nitrogen have a high growth rate. Furthermore, these cells contain high amounts of lipids.

Other articles, such as the one by Han Xu, Xiaoling Miao, Qingyu Wu, “High quality biodiesel production from a microalga Chlorella protothecoides by heterotrophic growth in fermenters”, Journal of Biotechnology, 126, (2006), 499-507, indicate that heterotrophic culture conditions, i.e. in the absence of light, make it possible to obtain an increased biomass with a high content of lipids in the microalgal cells.

The solid and liquid growth media are generally available in the literature, and the recommendations for preparing the particular media which are suitable for a large variety of microorganism strains can be found, for example, online at www.utex.org/, a website maintained by the University of Texas at Austin for its algal culture collection (UTEX).

In the light of their general knowledge and the abovementioned prior art, those skilled in the art responsible for culturing the microalgal cells will be entirely capable of adjusting the culture conditions in order to obtain a suitable biomass, preferably rich in lipids.

In particular, the fermentation protocol can be defined on the basis of that described entirely generally in patent application WO 2010/120923.

According to the present invention, the microalgae are cultured in liquid medium in order to produce the biomass as such.

The production of biomass is carried out in fermenters (or bioreactors). The specific examples of bioreactors, the culture conditions, and the heterotrophic growth and methods of propagation can be combined in any appropriate manner in order to improve the efficiency of the microbial growth and the lipids and/or of protein production.

In one particular embodiment, the fermentation is carried out in fed-batch mode with a glucose flow rate adjusted so as to maintain a residual glucose concentration of from 3 to 10 g/l.

During the glucose feed phase, the nitrogen content in the culture medium is preferably limited so as to allow the accumulation of lipids in an amount of 30%, 40%, 50% or 60%. The fermentation temperature is maintained at a suitable temperature, preferably between 25 and 35° C., in particular 28° C. The dissolved oxygen is preferably maintained at a minimum of 30% by controlling the aeration, the counter pressure and the stirring of the fermenter.

In one preferred embodiment, it was shown that the pH during the fermentation had an impact on the organoleptic quality of the final product. Thus, the initial pH of the fermentation is fixed between 6.5 and 7, preferably at a value of 6.8, and it is then regulated during fermentation at a value of between 6.5 and 7, preferably at a value of 6.8. The production fermentation time is from 3 to 6 days, for example from 4 to 5 days.

Preferably, the biomass obtained has a concentration of between 130 g/l and 250 g/l, with a lipid content of approximately 50% by dry weight, a fiber content of from 10% to 50% by dry weight, a protein content of from 2% to 15% by dry weight, and a sugar content of less than 10% by weight.

Next, the biomass obtained at the end of fermentation is harvested from the fermentation medium and subjected to the conditioning method as described above.

After the conditioning, the biomass is converted into microalgal flour. The principal steps for preparing the microalgal flour comprise in particular milling, homogenization and drying.

The microalgal flour can be prepared from the concentrated microalgal biomass which has been mechanically lyzed and homogenized, the homogenate then being spray-dried or flash-dried.

The biomass cells used for the production of microalgal flour are preferably lyzed in order to release their oil or lipids. The cell walls and the intracellular components are milled or reduced, for example using a homogenizer, to non-agglomerated cell particles or debris. Preferably, the resulting particles have an average size of less than 500 μm, 100 μm or even 10 μm or less.

The lyzed cells may also be dried. For example, a pressure disruptor can be used to pump a suspension containing the cells through a restricted orifice so as to lyze the cells. A high pressure (up to 1500 bar) is applied, followed by an instantaneous expansion through a nozzle. The cells can be broken by three different mechanisms: running into the valve, high shear of the liquid in the orifice, and a sudden drop in pressure at the outlet, causing the cell to explode. A Niro homogenizer (GEA Niro Soavi) (or any other high-pressure homogenizer) can be used to break cells. This treatment of the algal biomass under high pressure (approximately 1500 bar) generally lyzes more than 90% of the cells and reduces the size of the particles to less than 5μ. Preferably, the pressure applied is from 900 bar to 1200 bar, in particular 1100 bar.

In addition and in order to increase the percentage of lyzed cells, the microalgal biomass may undergo a high-pressure double treatment, or even more (triple treatment, etc.). Preferably, a double homogenization is used in order to increase the percentage of lyzed cells greater than 50%, greater than 75% or greater than 90%. The percentage of lyzed cells of approximately 95% has been observed by means of this double treatment.

Lysis of the microalgal cells is optional but preferred when a flour rich in lipids (in particular greater than 10%) is desired. Thus, the microalgal flour can optionally be in the form of non-lyzed cells.

Preferably, at least one partial lysis is desired, i.e. the microalgal flour is in the form of partially lyzed cells and contains from 25% to 75% of lyzed cells. Preferably, a maximum or even total lysis is desired, i.e. the microalgal flour is in the form of strongly or even totally lyzed cells and contains 85% or more of lyzed cells, preferably 90% or more. Thus, the microalgal flour is capable of being in a non-milled form up to an extremely milled form with degrees of milling greater than 95%. Specific examples relate to microalgal flours having degrees of milling of 50%, 85% or 95% of cell lysis, preferably 85% or 95%.

Alternatively, a ball mill may be used. In this type of mill, the cells are agitated in suspension with small abrasive particles. The breaking of the cells is caused by the shear forces, the milling between the beads, and the collisions with beads. In fact, these beads break the cells so as to release the cell content therefrom. The description of an appropriate ball mill is, for example, given in the patent U.S. Pat. No. 5,330,913.

A suspension of particles, optionally of smaller size than the cells of origin, is thus obtained in the form of an “oil-in-water” emulsion.

This emulsion can then be spray-dried and the water is eliminated, leaving a dry powder containing the cell debris and the lipids. After drying, the water content or the moisture content of the powder is generally less than 10%, preferentially less than 5% and more preferably less than 3% by weight.

Preferably, the microalgal flour is prepared in the form of granules. The microalgal flour granules are capable of being obtained by means of a particular spray-drying process, which uses high-pressure spray nozzles in a parallel-flow tower which directs the particles to a moving belt located in the bottom of the tower. The material is then transported as a porous layer through post-drying and cooling zones, which give it a crunchy structure, like that of a cake, which breaks up at the end of the belt. The material is then processed to obtain the desired particle size. In order to carry out the granulation of the algal flour, according to this spray-drying principle, a Filtermat™ spray-dryer sold by the company GEA Niro or a Tetra Magna Prolac Dryer™ drying system sold by the company Tetra Pak can be used for example.

In one preferred embodiment, subsequent to the conditioning method, the method for preparing the microalgal flour granules may comprise the following steps:

1) preparing an emulsion of microalgal flour with a solids content of between 15% and 40% by dry weight,

2) introducing this emulsion into a high-pressure homogenizer. This high-pressure homogenization of the emulsion can be accomplished in a two-stage device, for example a Gaulin homogenizer sold by the company APV, with a pressure of 100 to 250 bar at the first stage, and 10 to 60 bar at the second stage,

3) spraying in a vertical spray-dryer equipped with a moving belt at its base, and with a high-pressure nozzle in its upper part, while at the same time regulating:
a) the pressure applied at the level of the spray nozzles at values of more than 100 bar, preferably between 100 and 200 bar, and more preferably between 160 and 170 bar,
b) the input temperature between 150° C. and 250° C., preferably between 180° C. and 200° C., and
c) the output temperature in this spray-drying zone between 60° C. and 120° C., preferably between 60° C. and 110° C. and more preferably between 60° C. and 80° C.,

4) regulating the input temperatures of the drying zone on the moving belt between 40° C. and 90° C., preferably between 60° C. and 90° C., and the output temperature between 40° C. and 80° C., and regulating the input temperatures of the cooling zone at a temperature between 10° C. and 40° C., preferably between 10° C. and 25° C., and the output temperature between 20° C. and 80° C., preferably between 20° C. and 60° C.,

5) collecting the microalgal flour granules thus obtained.

According to the invention, the biomass extracted from the fermentation medium by any means known to those skilled in the art is then:
concentrated (for example by centrifugation),
optionally preserved by adding standard preservatives (sodium benzoate and potassium sorbate for example),
cellularly milled.

The invention will be understood more clearly from the examples which follow, which are intended to be illustrative and nonlimiting.

### EXAMPLES

### Example 1. Preparation of the Various Batches of Chlorella protothecoldes Biomass Flour

A. Description of the Standard Protocol: From Biomass Production to Flour Production

1. Fermentation

The fermentation protocol is adapted from the one described entirely generally in patent application WO 2010/120923.

The production fermenter is inoculated with a pre-culture of Chlorella protothecoides. The volume after inoculation reaches 9000 l.

The carbon source used is a 55% weight/weight glucose syrup sterilized at 130° C. for 3 minutes.

The fermentation is carried out in fed-batch mode with a glucose flow rate adjusted so as to maintain a residual glucose concentration of from 3 to 10 g/l.

The production fermentation time is from 4 to 5 days.

At the end of fermentation, the cell concentration reaches 185 g/l.

During the glucose feed phase, the nitrogen content in the culture medium is limited so as to allow the accumulation of lipids in an amount of 50%.

The fermentation temperature is maintained at 28° C.

The fermentation pH before inoculation is adjusted to 6.8 and is then regulated on this same value during the fermentation.

The dissolved oxygen is maintained at a minimum of 30% by controlling the aeration, the counter pressure and the stirring of the fermenter.

2. Biomass Conditioning

The fermentation must is heat-treated on a high temperature/short time (“HTST”) zone for 1 min at 75° C. and cooled to 6° C.

The biomass is then washed with decarbonated drinking water with a dilution ratio of 6 volumes of water for 1 volume of biomass, and concentrated by centrifugation using an Alfa Laval Feux 510.

The biomass is then acidified to pH 4 with 75% phosphoric acid and then preservatives are added (500 ppm sodium benzoate/1000 ppm potassium sorbate).

3. Biomass Milling

The biomass is then milled with a Netzsch LME500 ball mill using zirconium silicate balls 0.5 mm in diameter.

The degree of milling targeted is from 85% to 95%.

The product is kept cold throughout this process during the storage phases and by online cooling with dedicated exchangers.

Antioxidants are added (150 ppm/dry of ascorbic acid and 500 ppm/dry of a mixture of tocopherols) as prevention of degradation by oxidation.

The medium is adjusted to pH 7 with potassium hydroxide.

4. Drying the Flour

The product is then pasteurized at 77° C. for 3 minutes online with the drying operation.

The latter is carried out on a Filtermat FMD125 with a cyclone. The nozzle pressure is 160-170 bar.

B. Definition of the Sensory Panel and of the Descriptors Enabling the Evaluation of the Organoleptic Quality of the Microalgal Flours Obtained from the Biomass

A set of 14 individuals was thus brought together to evaluate the various biomass batches produced, using the following descriptors:

| Descriptors | | Reference |
|---|---|---|
| Appearance | Color (from light to dark) | |
| Texture | Coating | Whole milk + 5% cream |
| Savors | Sweet | 1% sucrose |
| Flavors | Mushroom | 100 g of mushrooms in 100 ml |
| | | of cold water /X 4 dilution |
| | Cereals | 10% Ebly Solution |
| | Butter/dairy product | |
| | Rancid oil | 1.5% Oxidized oil |
| | Vegetable aftertaste | Very unacceptable microalgal |
| | | flour |

The applicant company then found that the tasting matrix is advantageously constructed from the following formula:
7% of Microalgal flour
1% granulated sugar
0.25% household vanilla flavoring
91.75% skimmed milk.

The mixture is homogenized with an immersion mixer until a homogeneous mixture is obtained (approximately 20 seconds) and is then heated at 75° C. for 5 minutes in a water bath.

At each tasting session, 4 to 5 products are evaluated with regard to each descriptor in comparison with a microalgal flour reference batch 1.

All the products are evaluated one after the other, on scales ranging from 1 to 9 in the following way:
Value of 1: the descriptor evaluated is not present in the product.
Value of 5: the descriptor evaluated is present in the product in exactly the same way as on reference product 1.
Value of 9: the descriptor evaluated is very present in the product.

Reference batch 1 is a microalgal flour that complies in the sense that it has the sensory profile “satisfying” all these descriptors.

Preferably, reference batch 1 is not to be considered as the microalgal flour having the optimized sensory profile: it is a microalgal flour perceived by the sensory panel as “satisfactory”, in particular having a note of 5 on all the descriptors tested.

The other batches of microalgal flour will therefore be classified by the sensory panel on either side of this reference batch 1.

Preferably, a reference batch 2 considered to be “very unacceptable” since it does not satisfy the descriptors relating to the aromatic notes, in particular of Savors and Flavors, is also included. This batch is therefore as distant as possible from reference batch 1.

Analyses of variance (ANOVAs) are carried out in order to evaluate the discriminating capacity of the descriptors (descriptors of which the p-value associated with the Fisher test—type-3 ANOVA—is less than 0.20 for the Flour effect in the model descriptor ˜Flour+judge).

The Flour effect is interpreted as the discriminating capacity of the descriptors: if there is no effect (Critical Probability>0.20), the flours were not discriminated according to this criterion. The smaller this critical probability, the more discriminating the descriptor is.

A Principal Component Analysis (PCA) is then carried out in order to obtain sensory mapping of the flours, and a simultaneous representation of all the flours regarding all the descriptors.

Data Processing Software

The analyses were carried out using the R software (freely sold):

R version 2.14.1 (2011-12-22)

Copyright (C) 2011 The R Foundation for Statistical Computing

ISBN 3-900051-07-0

Platform: i386-pc-mingw32/i386 (32-bit)

The software is a working environment which requires the loading of modules containing the calculation functions.

The modules used in this study are the following:
For the PCA: Package FactoMineR version 1.19
For the ANOVA: Package car version 2.0-12

C. Impact of the Fermentation pH

The fermentation pH conditions are conventionally defined in the standard protocol starting from the premise that the fermentation pH should be fixed at a value of 6.8 (pH of the optimum growth known to those skilled in the art for microalgae of the Chlorella protothecoides genus), but without the impact of this pH value on the organoleptic properties of the microalgal flours being either studied or established.

Two series of flour batches are therefore produced from biomass, prepared at two neutral (6.8) and acidic (5.2) pH conditions. This value of 5.2 was chosen so as to take into account the bacteriological constraints (an acidic pH being relatively unfavourable to the growth of contaminating bacteria).

Table I below presents the references of the batches produced at these two pH values.

TABLE I

| batches | pH |
|---|---|
| batch 21 | 6.8 |
| batch 23 | 6.8 |
| batch 24 | 5.2 |
| batch 31 | 6.8 |
| batch 53 | 5.2 |
| batch 61 | 5.2 |
| batch 111 | 6.8 |
| batch 131 | 6.8 |

Each of these batches is then evaluated by the sensory panel according to the descriptors presented above.

The 8 different batches (batch 21, batch 23, batch 24, batch 31, batch 53, batch 61, batch 111 and batch 131) were analyzed according to the method described above.

Two examples regarding the “butter/dairy products” and “vegetable aftertaste” descriptors are presented here.

“vegetable aftertaste”: Analysis of variance table

| | Df | Sum Sq | Mean Sq | Value F | Pr (>F) |
|---|---|---|---|---|---|
| Flour | 9 | 109.693 | 12.1881 | 18.2423 | <2e−16 |
| Judge | 13 | 18.732 | 1.4409 | 2.1566 | 0.01298 |
| Residues | 185 | 123.603 | 0.6681 | — | |

“butter/dairy products”: Analysis of variance table

| | Df | Sum Sq | Mean Sq | Value F | Pr (>F) |
|---|---|---|---|---|---|
| Flour | 9 | 8.292 | 0.92131 | 1.4530 | 0.1699 |
| Judge | 13 | 8.235 | 0.63347 | 0.9991 | 0.4547 |
| Residues | 160 | 101.451 | 0.63407 | — | |

It appears that the critical probabilities associated with the Flour effect for the 2 descriptors studied are less than 0.2: the 2 descriptors are therefore discriminating. The critical probability is smaller with regard to the “vegetable aftertaste” descriptor than with regard to the “butter/dairy products” descriptor, which signifies that a greater difference is observed between the Flours with regard to the first criterion than with regard to the second.

Table II below sums up the critical probabilities obtained for the Flour and judge effects for all the descriptors.

TABLE II

| | Flour | Judge |
|---|---|---|
| Color | 1.62E−31 | 1.16^{E}−05 |
| vegetable | 1.60E−21 | 1.30^{E}−02 |
| aftertaste | | |
| Rancid oil taste | 4.00E−06 | 9.00^{E}−04 |
| Coating | 1.48E−05 | 1.63^{E}−02 |
| Cereals | 4.05E−04 | 1.94^{E}−07 |
| Mushrooms | 1.37E−03 | 5.66^{E}−05 |
| Sweet | 3.23E−03 | 4.02^{E}−04 |
| Dairy products | 1.70E−01 | 4.55^{E}−01 |

All the descriptors are discriminating; they are all kept for establishing the PCA.

Since the aromatic is an essential criterion of the flours, the PCA was carried out on the descriptors relating to the flavors only (mushroom, cereals, vegetable aftertaste, dairy product, rancid). The graphic representation of this PCA is FIGS. 1 and 2.

Since the first axis of the PCA summarises more than 75% of the information, it is the coordinates of the products on this axis which we use as “variable/classification”. This classification therefore clearly gives an account of the sensory distances between the products.

This method makes it possible to establish a classification of the organoleptic quality of various microalgal flours, which can be represented as follows:

batch 111>batch 31>batch 21>batch 23>reference batch 1>batch 131>batch 24>batch 53>batch 61>ref batch 2, with a clear separation between, on the one hand, batches 111, 31, 21, 23 and 131 and, on the other hand, batches 24, 53 and 61.

From an overall point of view, the panel judged batches 111, 31, 21, 23 and 131 to be acceptable and batches 24, 53 and 61 to be unacceptable.

These results therefore clearly illustrate the impact of the fermentation pH on the presence of an aftertaste totally unacceptable for the acceptability of the product.

At pH 5.2, the sensory profile systematically has a vegetable aftertaste, whereas at pH 6.8, the sensory profile is more neutral overall, without a significant vegetable aftertaste.

On first reading, it therefore appears that the controlling of the fermentation pH at a value of 6.8 is a key criterion for the preparation of a microalgal flour having a suitable, or even optimized, sensory profile (batch 111).

However, given the organoleptic variability of the batches produced at pH 6.8, it must be noted that the pH is not the only parameter responsible for the effects observed.

D. Measurement of the Impact of the Steps of Conditioning the Biomass Before Milling Thereof on the Organoleptic Quality of the Flour Produced

The influence of the two principal steps of conditioning (=pre-milling) the biomass before milling thereof, the HTST heat treatment and the washing, is also studied.

Starting from the same biomass produced at pH 6.8 according to step 1 of the standard method described above, the steps of conditioning the biomass were carried out according to 4 different combinations (FIG. 3).

Said steps enabled the production of 4 batches: No. 1 to 4:
Combination No. 1 is the control with neither HTST treatment nor washing.
Combination No. 2: washing alone
Combination No. 3: HTST alone
Combination No. 4: HTST before washing.

4 batches of flour are produced according to these 4 combinations. The remainder of the steps are common to each series and make it possible to condition the sample for the sensory analysis. Table III below presents the list of descriptors that are discriminating on this set of products (p-value less than 0.2 with regard to the Flour effect):

TABLE III

| | Flour | Judge |
|---|---|---|
| Coating | 2.30E−02 | 5.79E−03 |
| Color | 3.08E−02 | 5.30E−02 |
| Sweet | 1.22E−01 | 6.61E−02 |
| vegetable aftertaste | 1.69E−01 | 7.37E−01 |
| Butter/dairy | 3.74E−01 | 2.50E−03 |
| products | | |
| Mushroom | 3.88E−01 | 4.02E−02 |
| Cereal | 5.56E−01 | 3.33E−02 |

A Principal Component Analysis is carried out in order to represent the differences between the various flours produced (in comparison with a flour selected as Reference 1, i.e., as explained above, microalgal flour perceived by the sensory panel as “satisfactory”, in particular having a note of 5 on all the descriptors tested).

The results are presented in FIGS. 4 and 5.

It is observed that:
reference flour 1 is less sweet than the 4 batches produced, more colored, more coating;
flours 1 and 3 are both the sweetest; the washing is therefore an important step for ensuring the neutrality of the product;
flours 1 and 2 have an aftertaste, which shows the advantage of the HTST treatment. On these 2 flours, the panel notices a different aromatic, never previously encountered, described as follows: acidity of yogurt, “herbal”, bitteress, chemical, spicy, this being more intense/characteristic on flour 1 than on flour 2.

When a washing step is added after the HTST heat treatment operation, the sensory neutrality of the sample is improved, with a reduction in the sweet note.

The combination integrating the steps of HTST and then washing of the biomass before milling thus makes it possible to improve the organoleptic properties of the final product by eliminating a note characteristic of the “crude” biomass, by improving its neutrality and by reducing the sweet note.

Additional combinations were tested in order to refine the characterization of the sensory impact of the “pre-milling” method. FIG. 6

Here, the HTST and washing operations are inverted:

Combination 4: HTST then washing (same combination as above)

Combination 5: HTST after washing.

Table IV below presents the list of descriptors that are discriminating on this set of products (p-value less than 0.2 with regard to the Flour effect):

TABLE IV

| | Flour | Judge |
|---|---|---|
| color | 5.33E−04 | 0.05 |
| sweet | 0.01 | 0.64 |
| cereals | 0.06 | 0.61 |
| mushroom | 0.17 | 0.29 |
| coating | 0.25 | 0.64 |
| vegetable aftertaste | 0.43 | 0.89 |
| butter/dairy | 0.71 | 0.78 |
| products | | |
| rancid | 0.98 | 0.88 |

A Principal Component Analysis is carried out in order to represent the differences between the two different flours produced (still relative to the control: reference batch 1).

The results are presented in FIGS. 7 and 8.

When the two steps are inverted, the microalgal flour corresponding to the washing before HTST (Combination 5) has an aromatic which is stronger in terms of mushroom/cereals and sweet.

Furthermore, the panelists commented that the product was “spicy”.

Combination 4 is in this case preferred for its more neutral sensory profile more favourable in food applications.

E. Impact of the Heat Treatment Itself on the Quality of the Biomass

The heat treatment operation causes a cell deactivation which has an effect on the properties of the biomass.

The percentage cell deactivation (expressed as % of residual viable cells after 1 minute of heat treatment) as a function of the heat-treatment conditions is presented in FIG. 9.

For a heat treatment lasting 1 minute, a percentage deactivation greater than 90% is achieved starting from 50° C.

The cell deactivation is accompanied by a phenomenon of release of intracellular soluble materials into the extracellular medium. This phenomenon is probably linked to a parietal permeabilization.

A decrease in cell purity, linked to an increase in the solids content of the extracellular medium, is generally observed after heat treatment of the biomass (FIG. 10). The soluble materials released consist mainly of sucrose and, to a lesser extent, salts and proteins.

An experimental design in which the heat-treatment conditions are varied was produced.

The table below presents the batches produced while varying the heat-treatment (HTST) conditions.

| batches | Operating conditions |
|---|---|
| 42 | No HTST treatment |
| 23 | HTST 1 min at 75° C. |
| 43 | HTST 1 min at 65° C. |
| 45 | HTST 1 min at 50° C. |
| 46 | HTST 3 min at 95° C. |

Table V below presents the list of descriptors that are discriminating on this set of batches (p-value less than 0.2 with regard to the effect produced):

TABLE V

| | Flour | judge |
|---|---|---|
| Vegetable aftertaste | 0.09 | 0.93 |
| Color | 0.10 | 0.05 |
| coating | 0.18 | 0.61 |
| mushroom | 0.29 | 0.01 |
| butter/dairy | 0.31 | 0.52 |
| products | | |
| rancid | 0.44 | 0.90 |
| Cereals | 0.48 | 0.72 |
| Sweet | 0.54 | 0.01 |

The PCA is carried out in order to represent the differences between the various batches (FIGS. 11 and 12).

Few descriptors are discriminating with regard to this space produced since off-notes were perceived with regard to descriptors other than those of the evaluation.

Indeed, batches 42 and 45, in addition to having a darker color, are particularly bitter, spicy and fermented, leaving a metallic sensation in the mouth.

These 2 batches are the least heat treated (42 did not receive any HTST treatment and 45: 1 min at 50° C.).

Batch 46, for its part, has a vegetable aftertaste; the heat treatment for 3 min at 95° C. would therefore be unfavourable to the sensory quality of the product.

Batch 23 has an intermediate profile; the heat treatment for 1 min/65° C. (batch 43) is most favourable for obtaining a neutral sensory profile.

F. Impact of the Washing

In the same way as previously, the applicant company explored the coupling of these new optimized heat-treatment conditions with an optimized washing step, making it possible to entrain these extracellular soluble materials, in order to obtain improved organoleptic properties of the microalgal flours produced.

Various washing conditions were tested.

The table below presents the batches produced by varying the washing conditions (according to a water volume/biomass volume ratio).

| Batches | Operating conditions |
|---|---|
| 47 | No washing |
| 49 | Washing 6/1 (water/biomass) |
| 50 | Washing 1/1 (water/biomass) |
| 51 | Washing 3/1 (water/biomass) |

It will be noted that this experimental design makes it possible to analyze the impact by “increasing” washing (from the “least washed” to the “most washed”, batch 47<batch 50<batch 51<batch 49).

Table VI below presents the list of descriptors that are discriminating on this set of batches (p-value less than 0.2 with regard to the Flour effect):

TABLE VI

| | Flour | judge |
|---|---|---|
| color | 0.00 | 0.00 |
| coating | 0.02 | 0.19 |
| sweet | 0.10 | 0.01 |
| mushroom | 0.24 | 0.00 |
| cereals | 0.46 | 0.00 |
| vegetable aftertaste | 0.51 | 0.01 |
| butter/dairy products | 0.60 | 0.27 |
| rancid | 0.84 | 0.04 |

The PCA is carried out in order to represent the differences between the various batches. The results are represented in FIGS. 13 and 14.

This study clearly demonstrates the essential nature of the washing. Product 47, which is not washed, is sweeter and was judged unacceptable. The non-washed product (47) distinguishes itself from the others since it is sweeter and has a different, atypical taste.

The other products of this study have a similar sensory profile.

It will nevertheless be noted that a “simple” wash (just 1 volume of water per volume of biomass) leads to a product quality that is entirely suitable and, by the same token, an appreciable economic saving on the industrial scale (1 useful volume of water rather than 6 volumes of water per volume of biomass treated).

G. Impact of the Acidification During the Harvesting of the Biomass

One of the parameters which is not at all considered in the control of the steps responsible for the organoleptic quality of the flours produced is the effect of the protocol for stopping the fermentation.

Conventionally, when, at the end of fermentation, the pO₂ value goes back up, which is a sign of total consumption of the residual glucose, the end-of-fermentation protocol consists of the following steps:
Stopping the pH regulation,
Cooling the fermenter to a Tp<20° C.,
Reducing the stirring and the flow rate of air, and
Maintaining an air pressure on the dome.

A gradual drop in the initial fermentation pH (whether it is moreover fixed at 5.2 or at 6.8) to a pH dose to 4 generally occurs.

It was demonstrated by the applicant company that this acidification correlates with a secretion of lactic acid resulting from a metabolism limited in terms of O₂ supply.

This observation was therefore evaluated from a sensory point of view so as to measure the impact of the duration of the storage phase before conditioning of the biomass, said storage leading to this acidification.

Two batches are produced: with storage for a period of 8 hours (favoring acidification) and without storage.

Table VII below presents the list of descriptors that are discriminating on this set of products (p-value less than 0.2 with regard to the Flour effect).

TABLE VII

| | Flour | judge |
|---|---|---|
| Coating | 1.71E−02 | 7.50E−02 |
| vegetable aftertaste | 2.27E−02 | 6.29E−02 |
| mushroom | 2.35E−02 | 1.25E−02 |
| Cereal | 7.40E−02 | 7.30E−02 |
| Sweet | 9.41E−02 | 3.69E−01 |
| butter/dairy | 1.10E−01 | 7.27E−02 |
| products | | |
| Rancid | 2.61E−01 | 3.25E−01 |
| Color | 6.10E−01 | 4.29E−03 |

The PCA is carried out in order to represent the differences between the flours. The results are represented in FIGS. 15 and 16.

The 3 products (including Reference batch 1) are classified on an axis ranging from butter/dairy products/sweet to cereal/mushroom/vegetable aftertaste/coating:

The “without storage/acidification” batch is sweeter with more pronounced butter/dairy products. Reference 1 is more coating with cereal/mushroom/vegetable aftertaste aromatic; the “with storage/acidification” test lies between the two.

If the “with storage/acidification” and “without storage/acidification” tests are relatively compared, the “without storage/acidification” test is thus more “neutral”; it has fewer off-notes than the “with storage/acidification” test.

The sensory analysis therefore dearly demonstrates that a long storage phase coupled to this acidification phenomenon slightly degrades the sensory profile of the final product since a cereal/mushroom/vegetable aftertaste note of weak intensity appears.

### DESCRIPTION OF THE FIGURES

FIG. 1: Graphic representation of the various batches (cloud of points) of the PCA—Impact of the fermentation pH.

FIG. 2: Circle of correlation of the PCA representing the sensory profiles of the various batches—Impact of the fermentation pH.

FIG. 3: Pre-milling combinations tested.

FIG. 4: Graphic representation of the various batches (cloud of points) of the PCA—impact of the steps of conditioning the biomass before it is milled.

FIG. 5: Circle of correlation of the PCA representing the sensory profiles of the various batches—impact of the steps of conditioning the biomass before it is milled.

FIG. 6: Other pre-milling combinations tested.

FIG. 7: Graphic representation of the various batches (cloud of points) of the PCA—impact of the steps of conditioning the biomass before it is milled.

FIG. 8: Circle of correlation of the PCA representing the sensory profiles of the various batches—impact of the steps of conditioning the biomass before it is milled.

FIG. 9: Percentage of residual viable cells after 1 minute of heat treatment 25 as a function of the heat-treatment conditions.

FIG. 10: Comparison of the composition of the supernatant fraction extracted from the biomass before and after (HTST) heat treatment.

FIG. 11: Graphic representation of the various batches (cloud of points) of the PCA—impact of the heat treatment.

FIG. 12: Circle of correlation of the PCA representing the sensory profiles of the various batches—impact of the heat treatment.

FIG. 13: Graphic representation of the various batches (cloud of points) of the PCA—impact of the washing.

FIG. 14: Circle of correlation of the PCA representing the sensory profiles of the various batches—impact of the washing.

FIG. 15: Graphic representation of the various batches (cloud of points) of the PCA—impact of the acidification during the harvesting of the biomass.

FIG. 16: Circle of correlation of the PCA representing the sensory profiles of the various batches—impact of the acidification during the harvesting of the biomass.

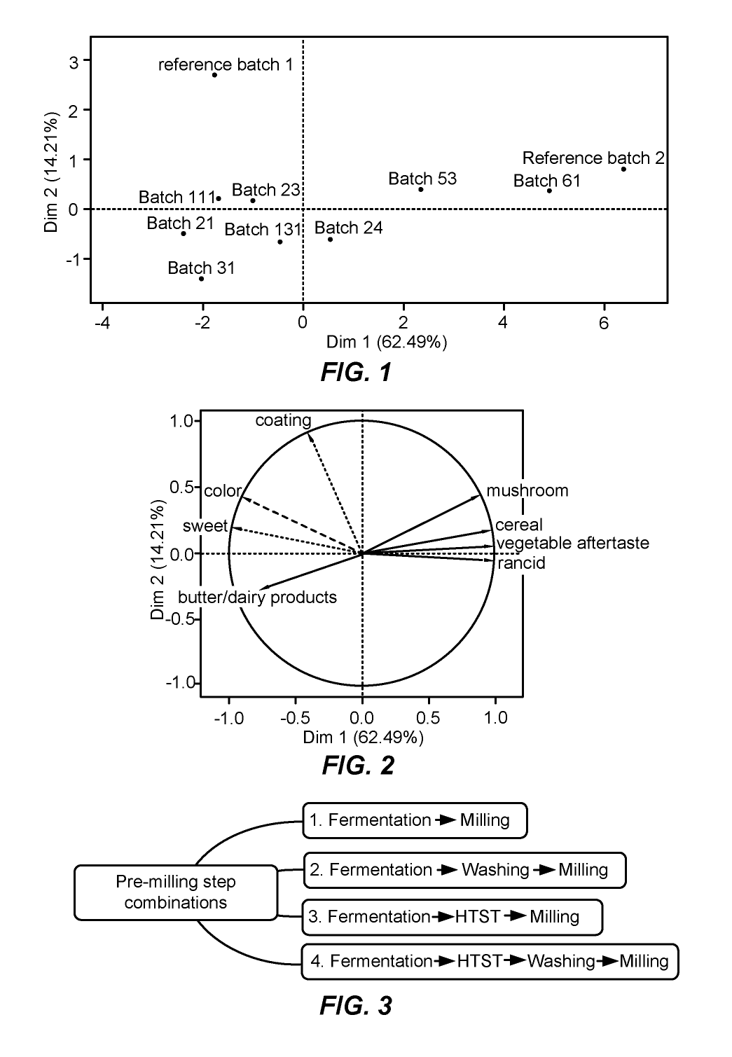

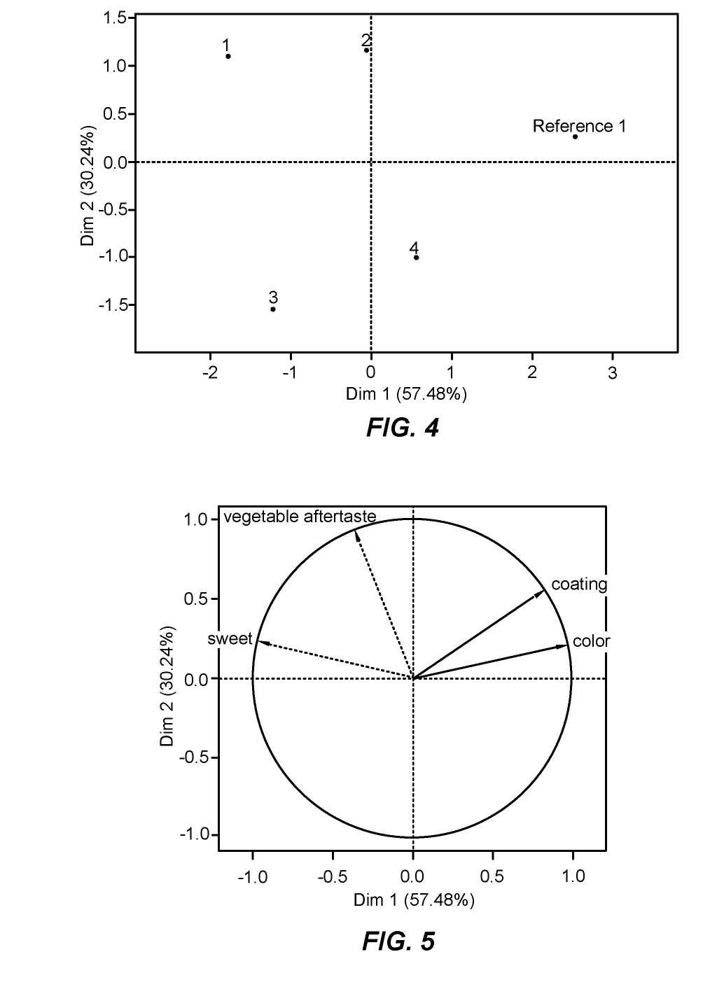

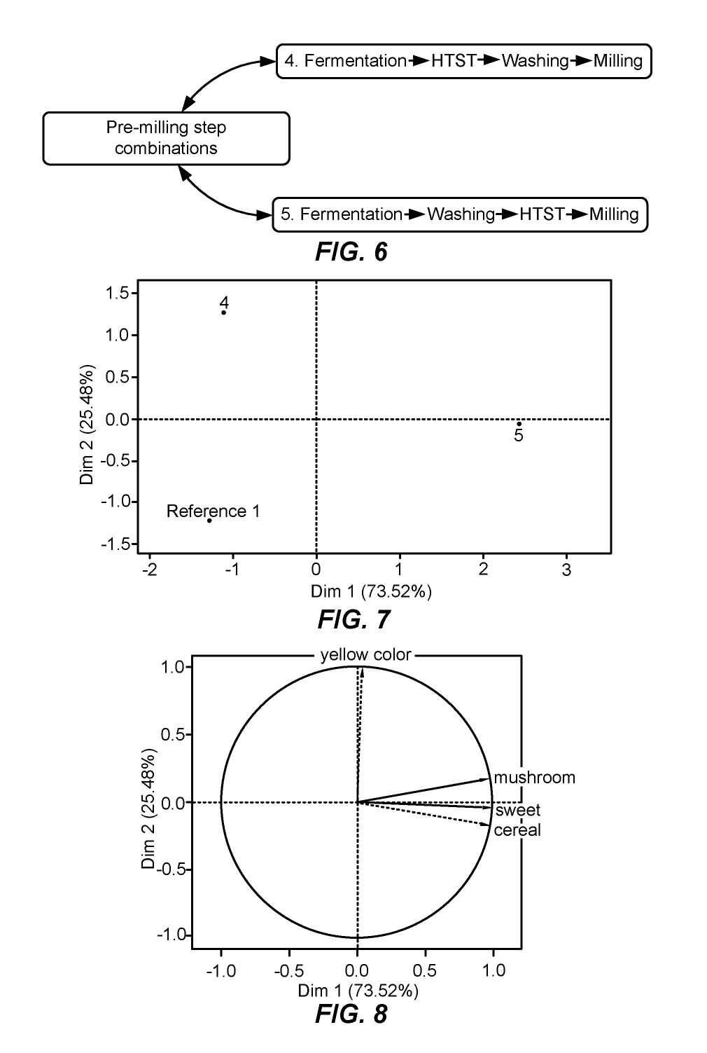

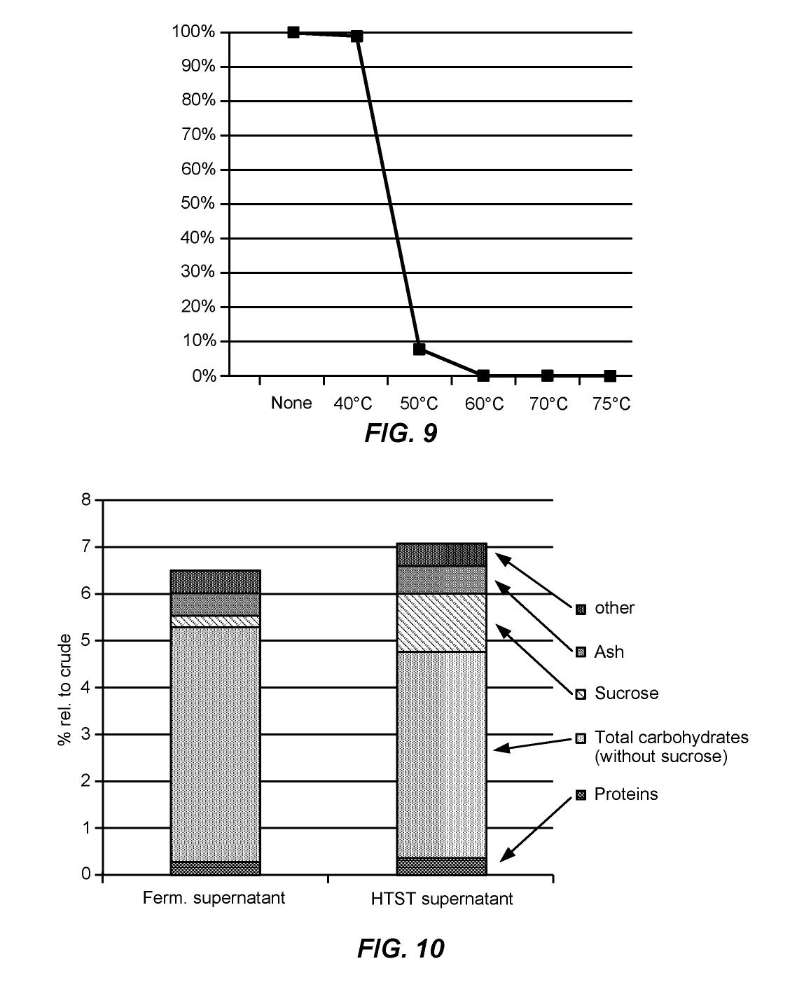

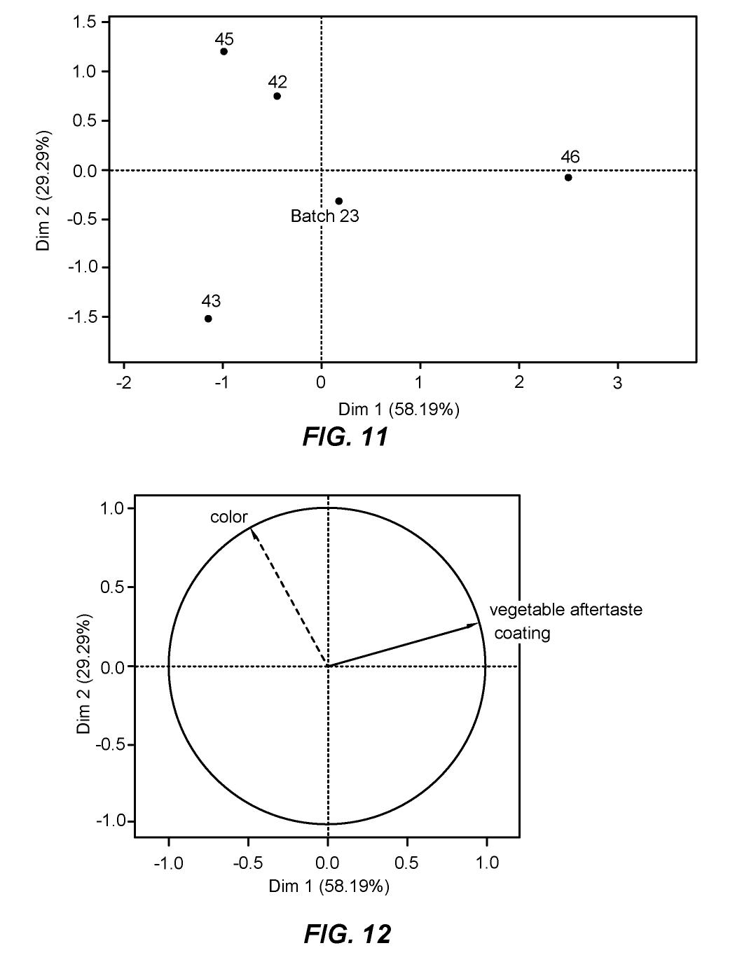

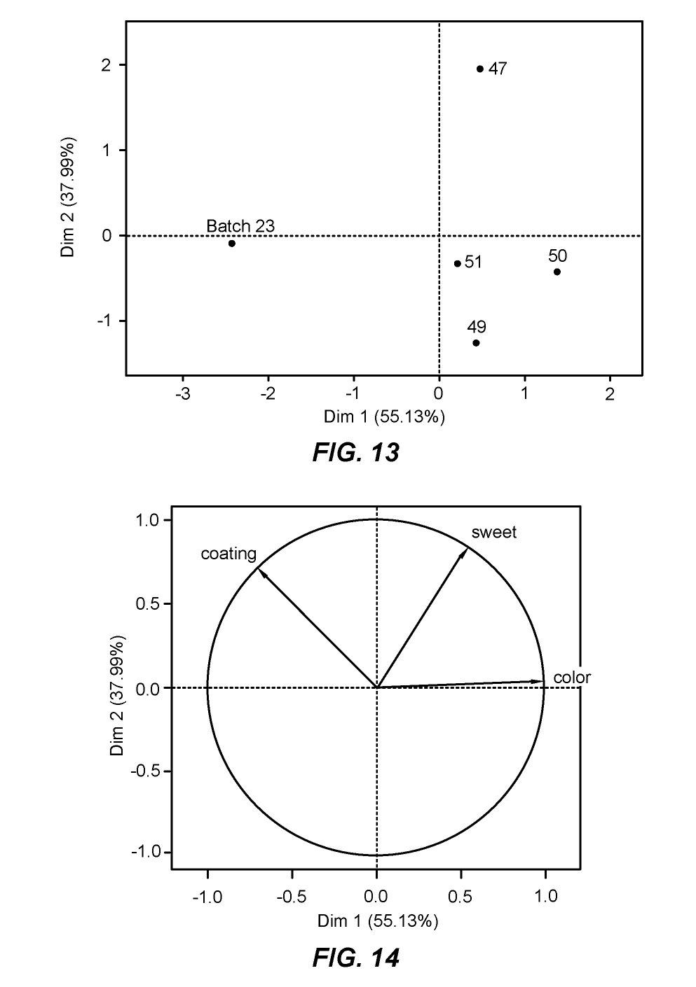

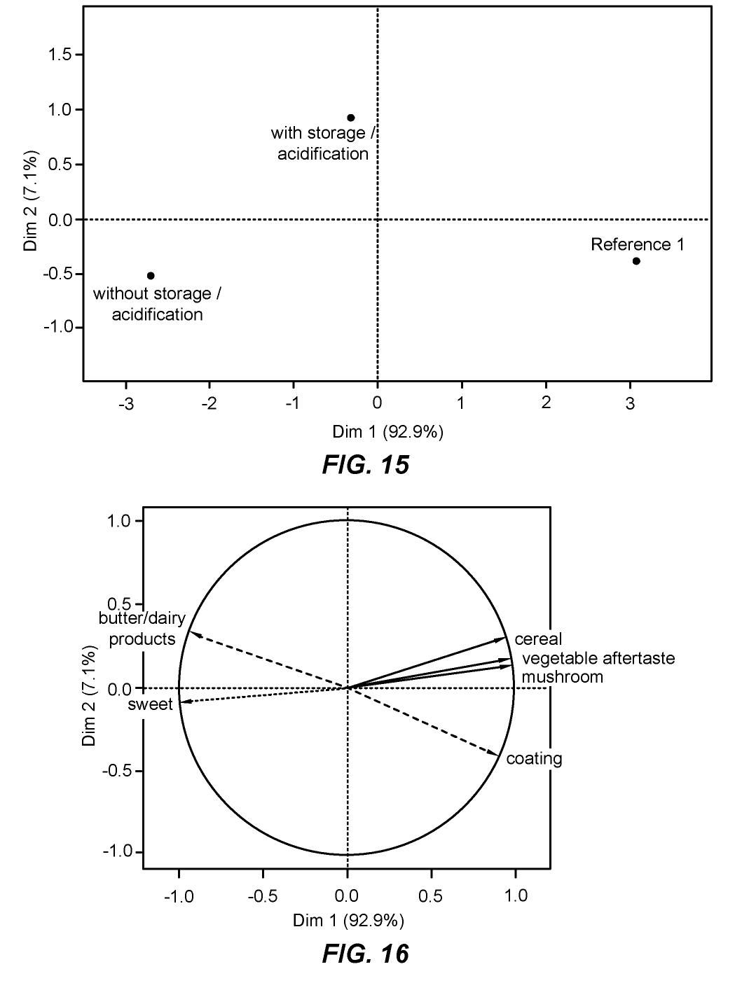

1.-9. (canceled)

10. A method for conditioning a biomass of microalgae of the Chlorella genus produced under heterotrophic conditions and in the absence of light for the preparation of a flour having an optimized sensory profile, the conditioning method comprising the steps of:
collection of the biomass directly at the end of fermentation, and storage for less than 8 hours before conditioning thereof,
high temperature/short time (HTST) heat treatment of the thus recovered and stored biomass, for 30 seconds to 1 min 30, at a temperature of between 60° C. and 68° C., and
washing of the HTST-treated biomass with at most 3 volumes of water per volume of biomass.

11. The method as claimed in claim 10, wherein the storage time for the biomass before it is conditions and milled is less than three hours.

12. The method as claimed in claim 10, wherein the HTST treatment is carried out for 1 minute at a temperature of 65° C.±2° C.

13. The method as claimed in claim 10, wherein the biomass is washed with one volume of water per volume of biomass.

14. The method as claimed in claim 10, wherein the HTST treatment is carried out before the step of washing the biomass.

15. The method as claimed in claim 10, wherein the biomass was obtained with fermentation of the microalgae of the Chlorella genus at an initial pH between 6.5 and 7 and with regulation of the fermentation pH at a value of between 6.5 and 7.

16. A method for producing a biomass of microalgae of the Chlorella genus for the preparation of a flour having an optimized sensory profile, comprising:
the production of a biomass by fermentation of microalgae of the Chlorella genus under heterotrophic conditions and in the absence of light, the initial pH of the fermentation and the regulation of the pH dring fermientation being fixed at a value of between 6.5 and 7; and
conditioning of the biomass, the conditioning comprising the steps of:
collection of the biomass directly at the end of fermentation, and storage for less than 8 hours before conditioning thereof,
high temperature/short time (HTST) heat treatment of the thus recovered and stored biomass, for 30 seconds to 1 min 30, at a temperature of between 60° C. and 68° C., and
washing of the HTST-treated biomass with at most 3 volumes of water per volume of biomass.

17. A method for preparing a flour of microalgae of the Chlorella genus having an optimized sensory profile, comprising:
conditioning a Chlorella biomass said conditioning comprising the steps of:
collection of the biomass directly at the end of fermentation, and storage for less than 8 hours before conditioning thereof,
high temperature/short time (HTST) heat treatment of the thus recovered and stored biomass, for 30 seconds to 1 min 30, at a temperature of between 60° C. and 68° C., and
washing of the HTST-treated biomass with at most 3 volumes of water per volume of biomass,
milling of the conditioned biomass; and
drying of the milled biomass.

18. The method as claimed in claim 17, said method comprising, prior to the conditioning, the production of said biomass by fermentation of microalgae of the Chlorella genus under heterotrophic conditions and in the absence of light, the initial pH of the fermentation and the regulation of the pH during fermentation being fixed at a value of between 6.5 and 7.
